(19) United States
(12) Patent Application Publication
(10) Pub. No.: US 2021/0330475 A1
(43) Pub. Date: Oct. 28, 2021

(54) ALIF Cage with Anchor Placement Options

(71) Applicant: Ingeniumspine, LLC, Phoenix, AZ (US)

(72) Inventors: Dennis CRANDALL, Mesa, AZ (US); B. Thomas BARKER, Bartlett, TN (US); Murali Murali KADABA, Austin, TX (US)

(73) Assignee: Ingeniumspine, LLC, Phoenix, AZ (US)

(21) Appl. No.: 17/122,701

(22) Filed: Dec. 15, 2020

Related U.S. Application Data
(63) Continuation of application No. 16/856,437, filed on Apr. 23, 2020.

(51) Int. Cl. A61F 2/44

(52) U.S. Cl. CPC A61F 2/4465; A61F 2220/0016; A61F 2250/0006

(57) ABSTRACT

An anterior lumbar interbody fusion device having a plurality of anchor apertures shaped and arranged in ways that provide options as to the number and locations of fixation points that anchor the device securely to the vertebrae. The anchor apertures may be angled towards or away from the cage vertical midline and cage horizontal midline, as well as angled differently relative to each other. In addition, the proximal opening of the anchor aperture is slightly wider than the distal opening, which permits the anchor to be positioned at a desired pitch within the opening, as opposed to being limited to a single entry angle. In a preferred embodiment, the proximal and distal opening of the anchor aperture are obround and each anchor aperture is sized to receive two anchors. In a given placement, anchors may be placed in some or all of the anchor apertures in each cage.

### CROSS-REFERENCE TO RELATED APPLICATIONS

This application is a continuation of co-pending U.S. application Ser. No. 16/856,437 filed Apr. 23, 2020.

### FIELD OF INVENTION

The present invention relates generally to devices for orthopedic surgery of the spine and particularly to devices for anterior lumbar interbody fusion which provide options for fixation directions and locations.

### BACKGROUND

The lumbar spine refers to the lower back, where the spine curves inward toward the abdomen. The curvature is known as lordosis. Anterior lumbar interbody fusion (“ALIF”) is a type of spine surgery that involves approaching the spine from the front (anterior) of the patient through the abdomen to remove all or part of a degenerated disc from between two adjacent vertebrae in the lower back. Once the disk is removed, a device known as an interbody cage or spacer is inserted into the disc space between the adjacent vertebrae to maintain spine alignment and intervertebral separation. The cage is anchored to the vertebrae using anchors such as screws or barbs. An anchor is inserted into each anchor aperture in the cage, passed partially through the cage, and secured into the vertebrae. The anchor apertures accommodate only one anchor and only in a specific location and angle of insertion, resulting in a pre-determined placement location. Additional surgical hardware such as rods, plates, hooks and wire may be used to support the vertebral structure during the healing process. Once healing is complete the adjacent vertebra are fused into a single monolithic structure.

Although the general shape of vertebrae are common between patients, the specific size, shape, lordosis, and condition of the cancellous bone are peculiar to each patient. These biological factors affect the size, shape and placement of the ALIF cage. The cage must be securely fixed to the vertebrae to ensure that it remains in the desired place during and after healing. Radiographs can give the surgeons a good idea of a given patient's condition prior to surgery, but sometimes the in vivo observation reveals unexpected factors, such as severe areas of necrotic, sclerotic, osteoporotic or cancerous bone as well as trajectories of pedicle screws from previous or current surgeries, which may prevent the surgeon from placing the anchor in a desired direction and location. The consequence of having predetermined anchor fixation locations in in combination with a lack of footing for the anchor is that the surgeon cannot anchor the device at the most secure fixation point(s). This induces risk that the patient will not heal properly or that the device may come loose over time. It would be advantageous to have a cage that permits the surgeon during surgery to have options for alternative locations to place the anchors in order to best secure the cage.

### SUMMARY OF THE INVENTION

This is an ALIF device having a plurality of anchor apertures in the cage body which are shaped and arranged in ways that provide options as to the number and locations of fixation points to anchor the device securely to the vertebrae.

The anterior facing surface has a vertical midline and a horizontal midline which intersect at a center point. The anchor apertures are arranged around the midlines such that the surgeon can choose to put anchors in only a subset of the anchor apertures and yet still attach the cage securely to the patient's vertebrae, avoiding undesirable amounts of skew about the center point or rotation about the vertical and horizontal midlines.

The anchor aperture is shaped inside to receive one or more anchors and prohibit the head of each anchor from passing through the anchor aperture. The anchor aperture is obround at its proximal end, comprising two semicircles connected by parallel lines tangent to their endpoints. The anchor aperture is also obround at its distal end. The radius of the semicircle at each proximal end is larger than the radius of the semicircle at each distal end, which permits the anchor to be positioned at a desired pitch and yaw within the opening, as opposed to being limited to a single entry angle. The anchor apertures may be angled radially towards or away from the cage vertical midline and cage horizontal midline, as well as angled differently relative to each other. In a preferred embodiment, each anchor aperture is sized to receive two anchors. In a given placement, anchors may be placed in some or all of the anchor apertures in each cage.

### BRIEF DESCRIPTION OF THE DRAWINGS

FIG. 1 is a top perspective view of a 4×1 aperture ALIF cage of the present invention with no anchors.

FIG. 2 is a front view of the ALIF cage of FIG. 1.

FIG. 3 is a top perspective view of a 4×1 aperture ALIF cage of the present invention with an anchor in the first aperture.

FIG. 4 is a front view of the ALIF cage of FIG. 3.

FIG. 5 is a rear cross-sectional view of the ALIF cage along line 5-5 of FIG. 1.

FIG. 6 is a cross-sectional view along line 6-6 of the ALIF cage FIG. 4.

FIG. 7 is a cross-sectional view along line 7-7 of the ALIF cage FIG. 4.

FIG. 8 is a top perspective view of a 4×2 aperture ALIF cage of the present invention with no anchors.

FIG. 9 is a front view of the ALIF cage of FIG. 8.

FIG. 10 is a top perspective view of a 4×2 aperture ALIF cage of the present invention with an anchor in the first lower aperture.

FIG. 11 is a front view of the ALIF cage of FIG. 10.

FIG. 12 is a rear cross-sectional view of the ALIF cage along line 12-12 of FIG. 8.

FIG. 13 is a cross-sectional view along line 13-13 of the ALIF cage FIG. 11.

FIG. 14 is a cross-sectional view along line 14-14 of the ALIF cage FIG. 11.

FIG. 15 is a front view of a 3×2 aperture ALIF cage of the present invention with anchors in each aperture.

FIG. 16 is a rear view of the 3×2 aperture ALIF cage of FIG. 15.

FIG. 17 is a top view of the 3×2 aperture ALIF cage of FIG. 15.

FIG. 18 is a side view of the 3×2 aperture ALIF cage of FIG. 15.

FIG. 19 is a top perspective view of the 3×2 aperture ALIF cage of FIG. 15.

FIGS. 20A-G illustrate the anchor placements available for the 3×2 aperture embodiment of the cage.

FIGS. 21A-I illustrate the anchor placements available for the 4×2 aperture embodiment of the cage.

FIG. 22 is a schematic illustration of a 3×2 aperture cage attached to lumbar vertebrae L4 and L5 using three anchors.

FIG. 23 is a schematic illustration of a 4×2 aperture cage attached to lumbar vertebrae L4 and L5 using four anchors.

FIG. 24 is a schematic illustration of a 2-anchor aperture with partial views of the two anchors.

### DETAILED DESCRIPTION OF THE INVENTION

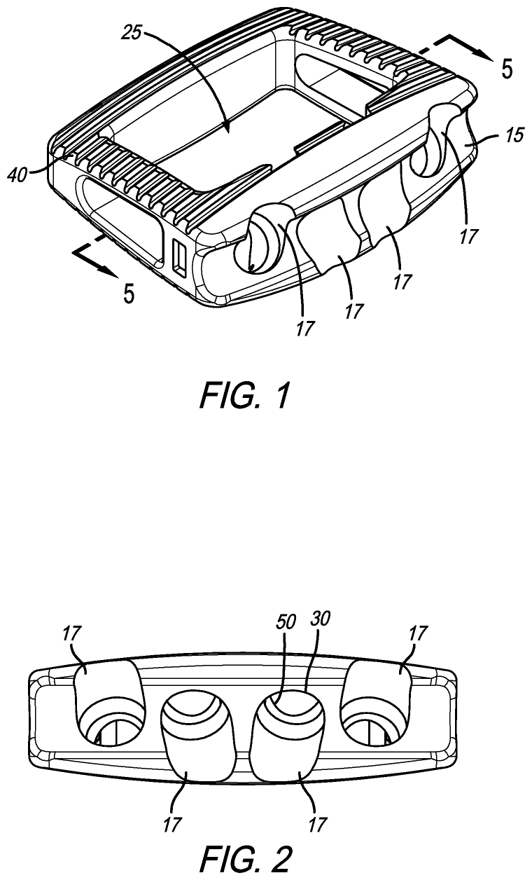

Each ALIF cage comprises a cage body 11 surrounding a central cavity 25. The shape of the cage body 11 as viewed from the top is preferably trapezoidal, but oval, D-shaped and other polygonal shapes will suffice. See FIG. 1. The cage body 11 has an anterior facing surface 15 and a cavity-facing surface 19, also referred to herein as front and back, respectively. The anterior facing surface 15 has a vertical midline 12 and a horizontal midline 14 which intersect at a center point 16. A plurality of anchor apertures 17 are through-holes in the cage body 11. In addition to anchor apertures 17, the cage body may have additional through-holes which are cut-outs used to reduce the volume of material used to make the cage body, but which are not situated in positions to enable anchors to be secured to the vertebrae.

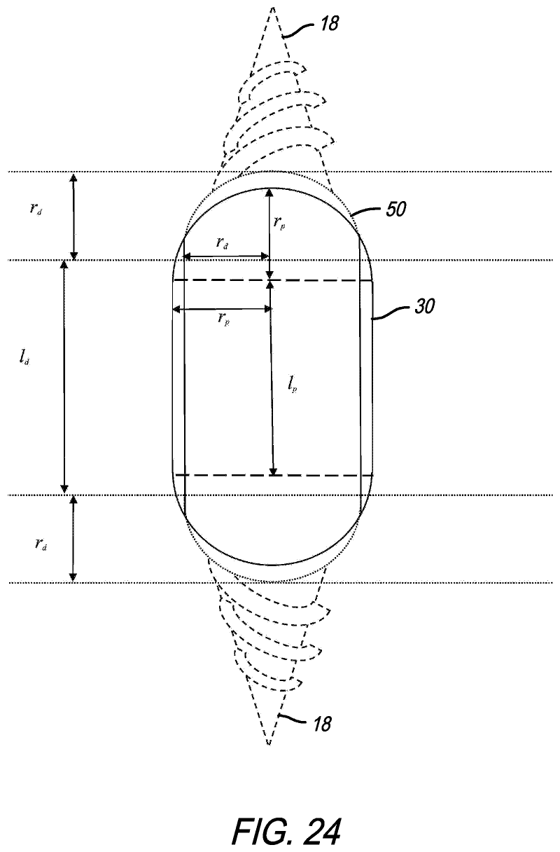

Each anchor aperture 17 is a through-hole having an entrance, namely a proximal opening 30 on the anterior-facing surface and an exit, namely a cavity-facing distal opening 50. The proximal opening 30 is an obround, also known as a discorectangle or having the shape of a stadium. The proximal obround 30 comprises two semicircles of radiusₚ connected by two parallel lines each of the lengthₚ. The distal opening 50 is also an obround, comprising two semicircles of radius_{d} connected by two parallel lines each of the lengths. See FIG. 24, which shows proximal and distal openings, radii, lengths, and shanks of the anchors 18, but not the anchor heads. The interior surface of the anchor aperture 17 is continuous, and the transition from the proximal opening to the distal opening can be a bevel, a chamfer, an L-shape, but is typically a fillet. See, for example, FIG. 13.

Radiusₚ is greater than radius_{d}, so the proximal opening 30 permits an anchor having an anchor head 21 of radius_{anchorhead} to be inserted through and past the proximal opening 30. Because the proximal opening 30 is wider than the distal opening 50, the anchor 18 may be positioned at a desired pitch and yaw within the proximal opening, as opposed to being limited to a single entry angle. This designed tolerance effectively creates a range of insertion angles, and permits the anchor to be inserted and placed at a wide range of positions. The range of insertion angle is typically 40 deg included angle. This is in contrast to prior art anchor apertures in ALIF devices in which the radius of the anchor aperture is constant from entry to exit, to serve as a physical guide to position the anchor in the vertebrae in only a single pitch and yaw, and therefore in only a single placement in the vertebrae. The radius of the anchorhead r_{anchorhead} is greater than the radius_{d}, so the anchor head cannot pass through the distal opening 50.

In addition to the designed variability in the insertion angle within a given aperture, the anchor apertures may be disposed in the cage body at different angles relative to the vertical midline 12, the horizontal midline 14, and to each other. The yaw of the anchor aperture 17 is defined as the angle an aperture is disposed relative to the vertical midline 12, either away from or toward it. The pitch of the anchor aperture is defined as the angle an aperture is disposed at an angle relative to the horizontal midline 14, either away from or toward it. In some embodiments the anchor aperture 17 is both yawed and pitched. Each anchor aperture may be angled differently relative to each other.

The cage body 11 has one or more anchor apertures 17 to receive the anchors, and preferably two or more anchor apertures. The anchor apertures 17 are arranged around the midlines such that the surgeon can choose to put anchors in only a subset of the anchor apertures and yet still attach the cage securely to the patient's vertebrae, avoiding undesirable amounts of skew about the center point or rotation about the vertical and horizontal midlines. In the preferred embodiments, the anchor apertures 17 are arranged in a row, spaced apart uniformly and symmetrically around the vertical and horizontal midlines. If an anchor aperture is centered on the vertical midline, it is sometimes referred to as being in the middle.

In other embodiments, the anchor apertures 17 are spaced non-uniformly in the cage body 11. When the apertures are not in uniformly spaced-apart alignment, the surgeon uses additional care in placing anchors in anchor apertures using the appropriate number of anchors and location(s) of fixation points to secure the cage body and prevent undesirable amounts of skew about the center point or rotation about the vertical and horizontal midlines.

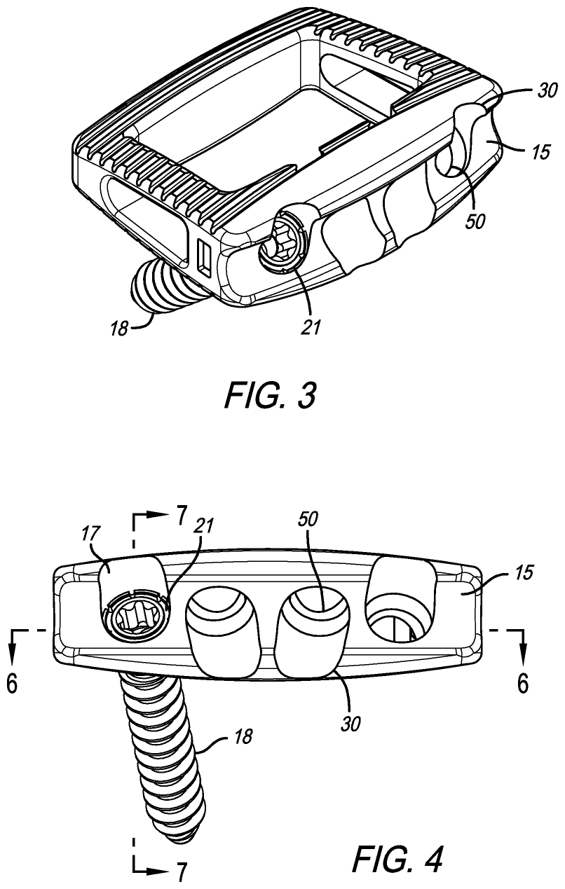

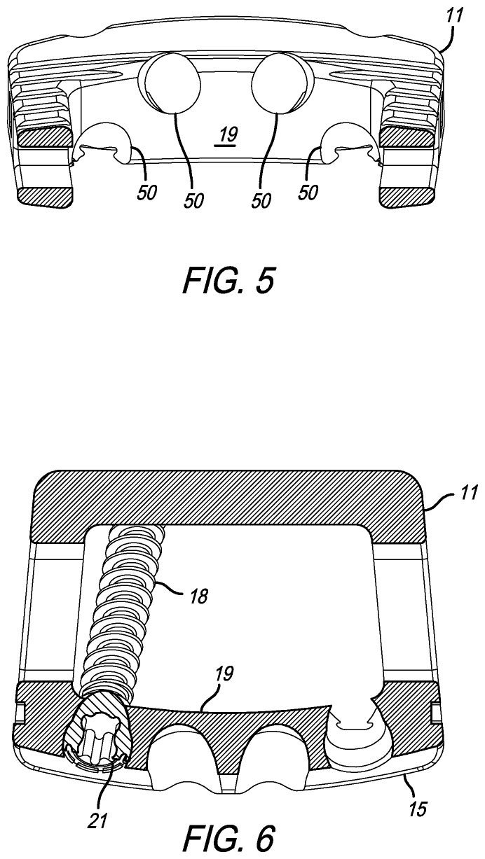

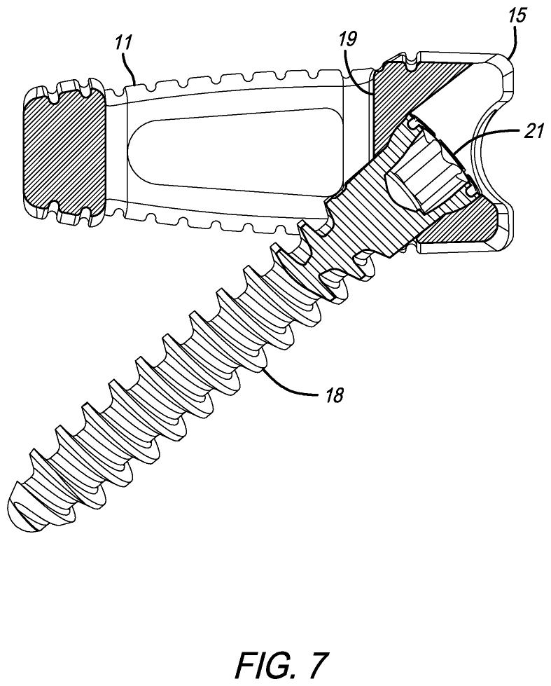

FIGS. 1-7 illustrate a cage body 11 having four anchor apertures 17, each of which can accept only a single anchor 18. This configuration is referred to as a 4×1 cage. The distal opening 50 has a smaller radius r_{d} than the proximal opening 30 so that the head of the anchor cannot pass completely through the anchor aperture 17. The length lₚ of the proximal obround is zero, so the semicircles of radius rₚ meet and form a circle. FIGS. 3, 4, 6 and 7 show a single anchor 18 in the far left anchor aperture. FIG. 6 shows the anchor aperture 17 is angled toward the vertical midline 12, resulting in the anchor 18 also being yawed toward the vertical midline 12. FIGS. 3 and 7 show the anchor aperture 17 is angled away from the horizontal midline 14, resulting in the anchor being pitched away from the horizontal midline 14 and out of the cage body 11.

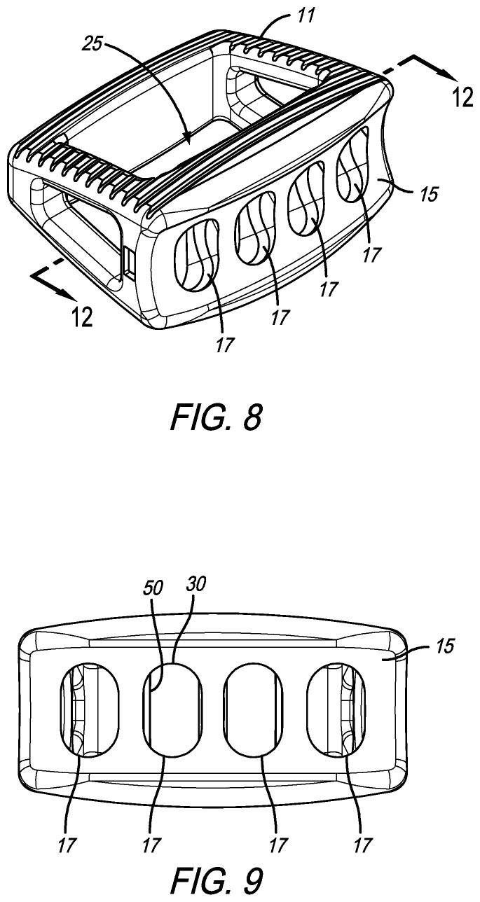

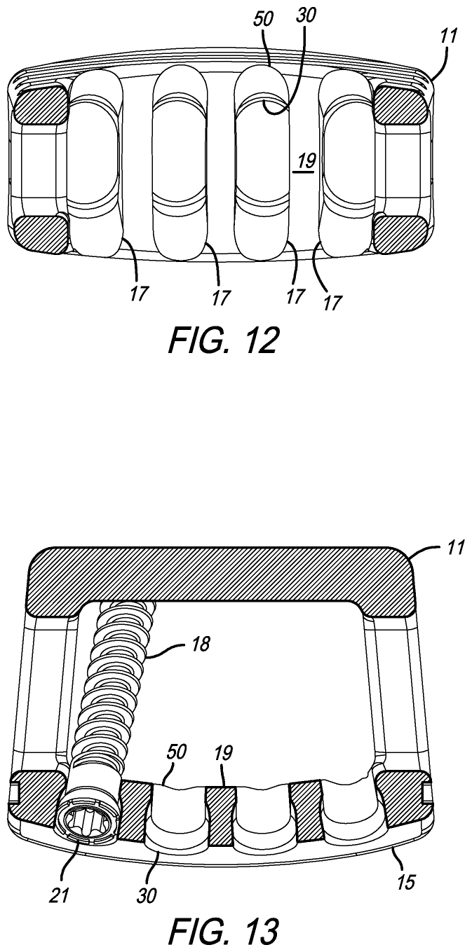

FIGS. 8-14 illustrate a cage body 11 having four anchor apertures 17, each of which can accept up to two anchors 18. This configuration is referred to as a 4×2 cage. The distal opening 50 has a smaller radius r_{d} than the proximal opening 30 so that the head of an anchor cannot pass completely through the anchor aperture 17. The length_{d} of the distal obround 50 is at least 4 times the radius of the anchor head r_{anchorhead}, so that two anchors 18 may be seated against the distal opening 50. Preferably the radius of the proximal opening rₚ is larger than the radius of the distal opening r_{d}, and the length of the proximal obround lₚ is shorter than the length of the distal opening I_{d}.

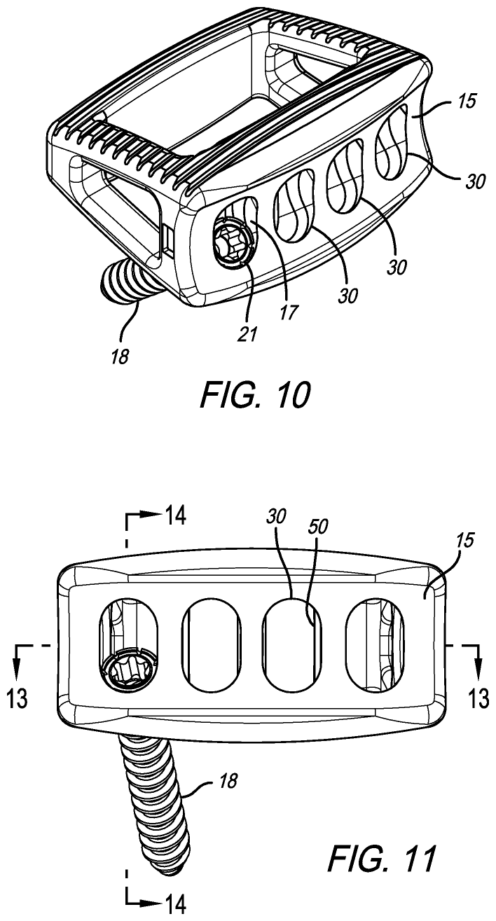

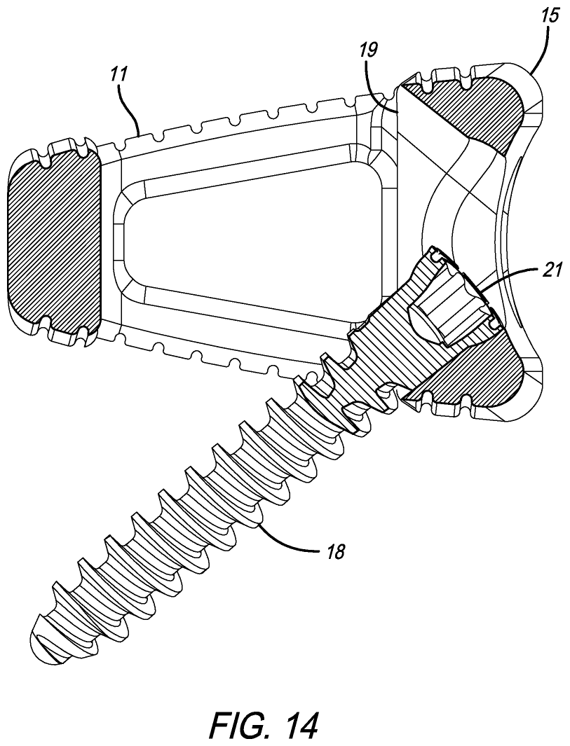

FIGS. 10, 11, 13 and 14 show a single anchor 18 in the far left anchor aperture, with room to place a second anchor (not shown). FIG. 13 shows the anchor aperture is angled toward the vertical midline, resulting in the anchor also being yawed toward the vertical midline. FIGS. 10 and 14 show the aperture is angled away from the horizontal midline, resulting in the anchor being pitched away from the horizontal midline and out of the cage body.

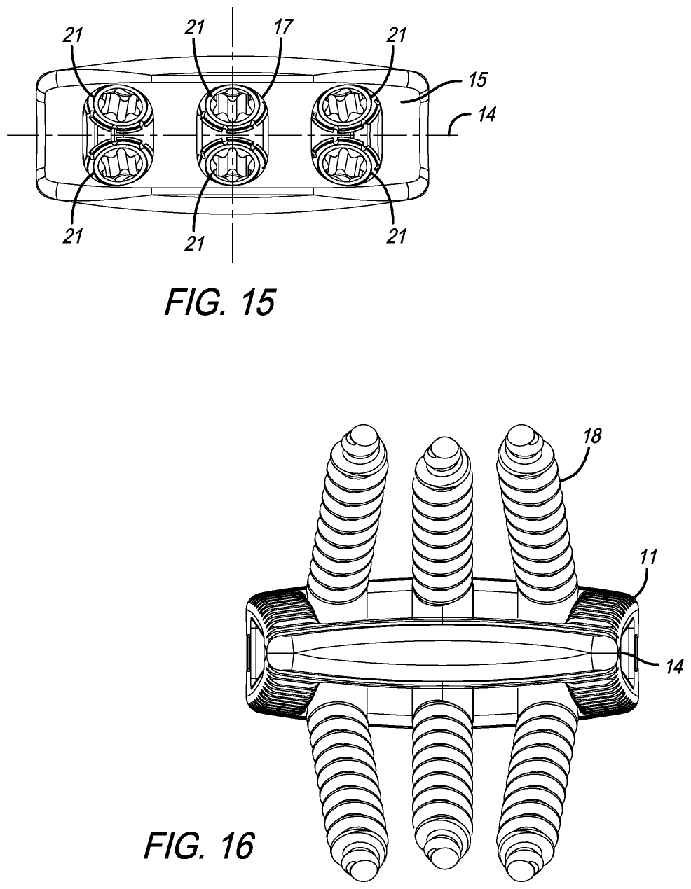

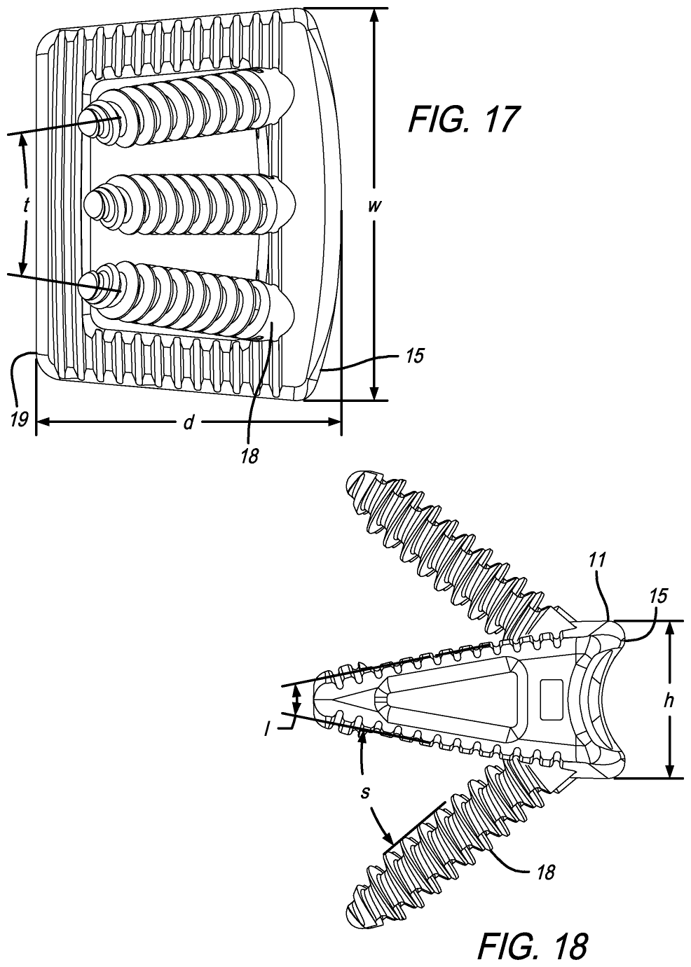

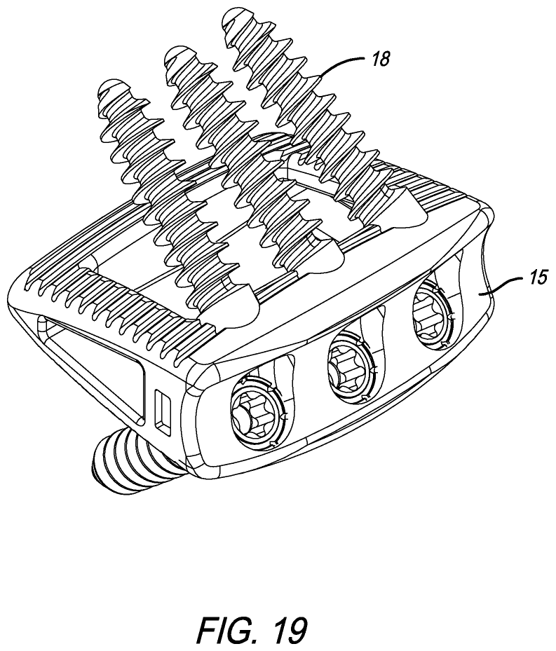

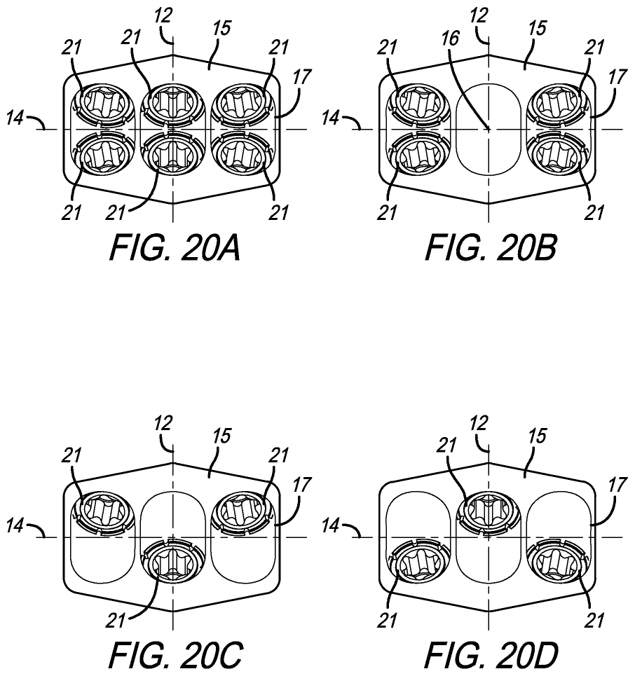

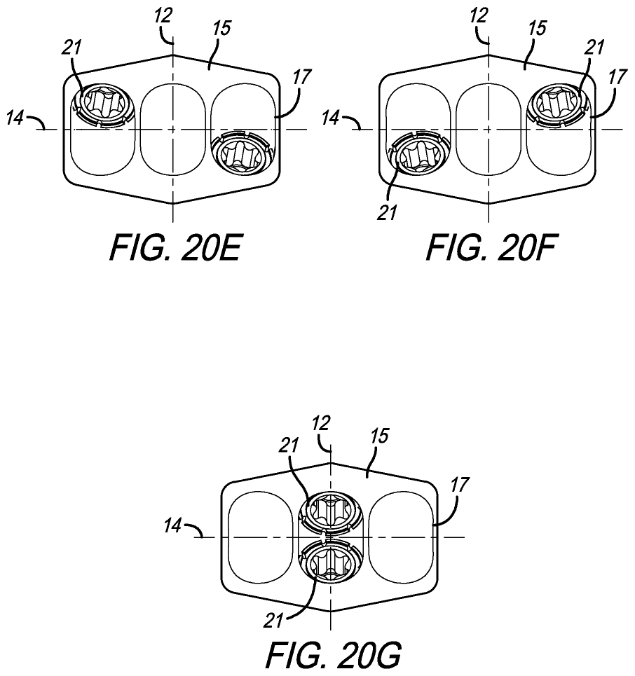

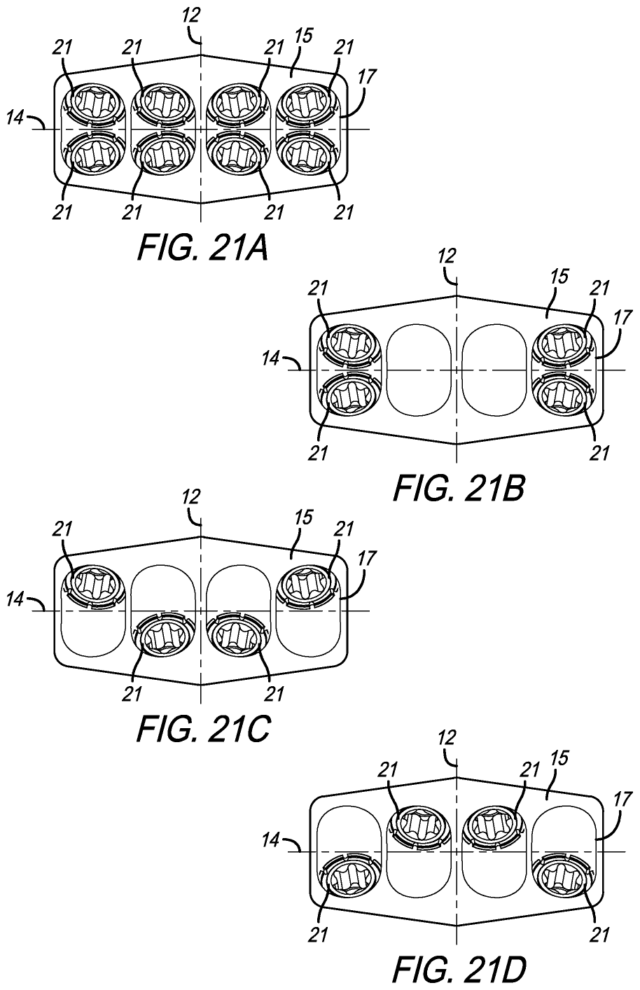

FIGS. 15 and 20A are front views of a cage body 11 with three anchor apertures 17, with two anchors 18 inserted into each anchor aperture 17. FIG. 19 shows a perspective view of the same. This configuration is referred to as a 3×2 cage. FIG. 16 is a rear view of a 3×2 cage with anchors 18 inserted into all possible locations. The width w of the cage and the radius of the anchorhead r_{anchorhead} determine how many anchor apertures 17 can fit in the cage body 11. See FIG. 17. The larger the width, the more anchor apertures 17 can fit. The height h of the cage determines how long the anchor apertures 17 can be. See FIG. 18. The larger the height, the longer the anchor apertures 17 can be. A shorter anchor aperture 17 permits only a single anchor 18, whereas a longer anchor aperture may be permit up to two anchors 18. The depth d of the cage body is the size of the cage body 11 measured from front 15 to back 19. See FIG. 17. The lordosis angle l describes the pitch of the cage body 11, which is a factor in the lordosis of the patient after the cage body 11 is secured in the vertebrae. The larger the lordosis angle l, the more lordosis of the patient's spine. See FIG. 18. The anchor apertures may be toed-in or toed-out, providing yaw, and the degree of such toe angle is indicated by angle t. See FIG. 1C. The angle of the anchor 18 out of the cage 11 is indicated as angle s. See FIG. 17. Angle s varies between 25 to 45 degrees and is typically 40 degrees.

In addition to the 4×1, 4×2 and 3×2 configurations shown in the drawings, 1×2, 2×2, 3×1 configurations are contemplated.

In addition to the proper choice of cage based on the physical size and lordosis of the vertebrae, the present cages enable the surgeon to pick and choose how many anchors s/he wants to use to anchor the cage body and the locations of the fixation points in the vertebrae. FIGS. 20A-G schematically illustrate the various number and locations that anchors 18 may be placed in a 3×2 cage to secure it to the vertebrae. For clarity, only the anchor head 21 is shown for each anchor 18; indicia are used on all the anchor heads 21, but on only one anchor aperture 17. To secure the cage to the patient, six or fewer anchors may be used, in various arrangements.

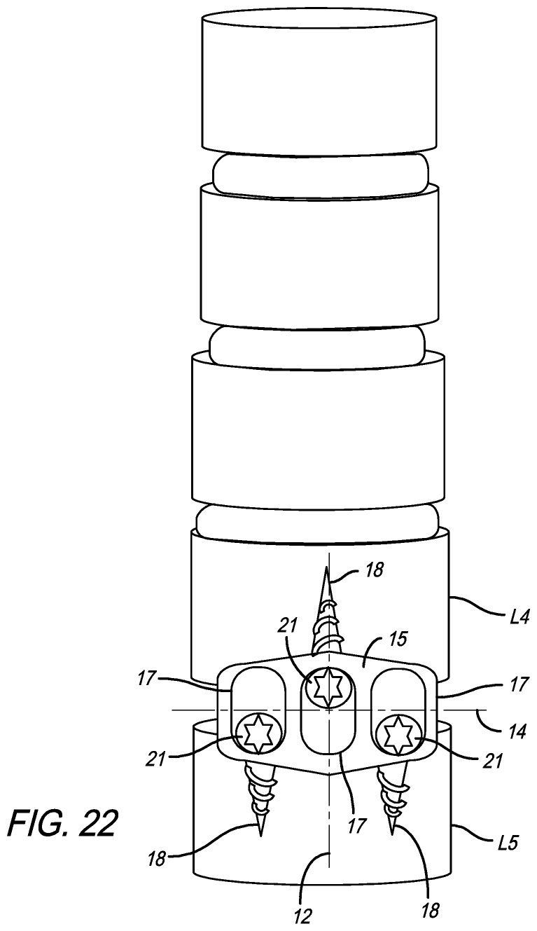

For ease in describing the placement of the anchors, the potential anchor placement locations are described in columns and rows. FIG. 20A shows a cage complete with six anchors 18 in three anchor apertures 17: anchors in column 1, rows 1 and 2; column 2, rows 1 and 2; and column 3, rows 1 and 2. FIG. 20B shows four anchors in two anchor apertures: in column 1, row 1; column 1 row 2; column 3, row 1; and column 3, row 2. FIG. 20C shows three anchors in three anchor apertures: in column 1, row 1; column 2, row 2; and column 3, row 1. FIG. 20D is the reciprocal anchor placement to that of FIG. 20C and uses three anchors, one each in three anchor apertures in column 1, row 2; column 2, row 1; and column 3, row 2. FIG. 20E shows two anchors in two anchor apertures: in column 1, row 1 and column 2, row 2. FIG. 20F is the reciprocal placement to that of FIG. 20E, and uses two anchors in two anchor apertures in column 1, row 2 and column 2, row 1. FIG. 20G shows two anchors in only a single anchor aperture: in column 2, row 1 and column 2, row 2. FIG. 22 shows a 3×2 cage attached to vertebrae with only three anchors, although up to six anchors could be used.

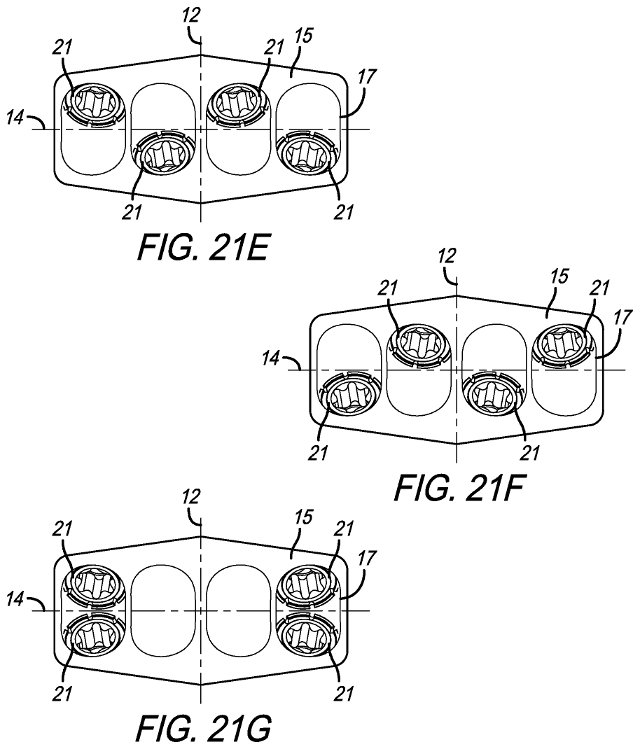

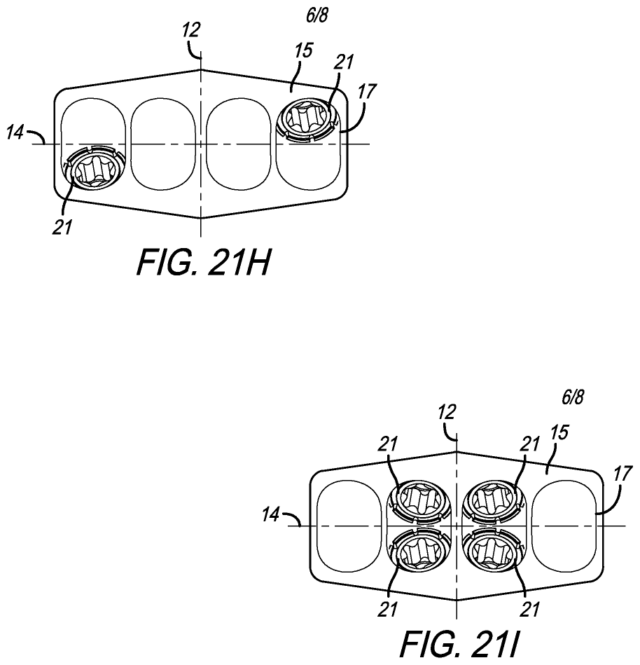

FIGS. 21A-I schematically illustrate the anchor placements available for the 4×2 cage to secure it to the vertebrae. For clarity, only the anchor head 21 is shown for each anchor 18; indicia are used on all the anchor heads, but on only one anchor aperture 17. To secure the cage to the patient, eight or fewer anchors may be used, in various arrangements. FIG. 21A shows eight anchors in all four anchor apertures. FIG. 21 B shows anchors in two anchor apertures: in column 1, row 1; column 1 row 2; column 4, row 1; and column 4, row 2. FIG. 21C shows four anchors in four anchor apertures: in column 1, row 1; column 2, row 2; column 3, row 2; and column 4, row 1. FIG. 21D is the reciprocal anchor placement to that of FIG. 21C and uses four anchors in four anchor apertures: in column 1, row 2; column 2, row 1; column 3, row 1; and column 4, row 2.

FIG. 21E shows four anchors in four anchor apertures: in column 1, row 1; column 2 row 2; column 3 row 1; and column 4, row 2. FIG. 21F is the reciprocal placement to that of FIG. 3E, and uses four anchors in four anchor apertures: in column 1, row 2; column 2 row 1; column 3 row 2; and column 4, row 1. FIG. 21G shows two anchors in two anchor apertures: column 1, row 1; and column 4, row 2. FIG. 21H the reciprocal anchor placement to that of FIG. 21G and shows two anchors in two anchor apertures: column 1, row 2; and column 4, row 1. FIG. 21I shows four anchors in two anchor apertures: in column 2, row 1; column 2, row 2; column 3, row 1, and column 3, row 2.

In some embodiments the cage body 11 is textured with ridges and rough surface finish on its top and bottom surfaces to provide increased surface area for improved binding to the vertebrae it is placed between, which aids in healing and keeping the cage body 11 in place. The drawings show a surface texture featuring spaced-apart ridges 40, but other types of surface texture features may suffice such as pebbles, teeth, serrations, and cross-hatching.

Each ALIF cage is preferably manufactured using additive manufacturing techniques, also referred to in the art as 3-D printing, followed by machining and surface finishing. Each cage is made of a material that provides the desired stiffness and strength. In a preferred embodiment the material is titanium or a titanium alloy such as Ti-6AI-4V. Polyether ether ketone (PEEK) and other bio-compatible materials may suffice.

Although the human lumbar vertebrae have a generally common shape from patient to patient, in practice the vertebrae differ in shape, size, spacing, and lordosis. Therefore, the cages needed for interbody fusion are ones that are best suited to each patient. A set of cage bodies of different ranges of width, depth, height and lordosis are supplied to the surgeon in the operating room so that the surgeon can select the desired cage body after first reviewing radiographs, CT/MRI scans and further determining the size by measuring the patient's intervertebral space in vivo. The size and shape of the cage body is chosen to best fit the intervertebral space in a given patient.

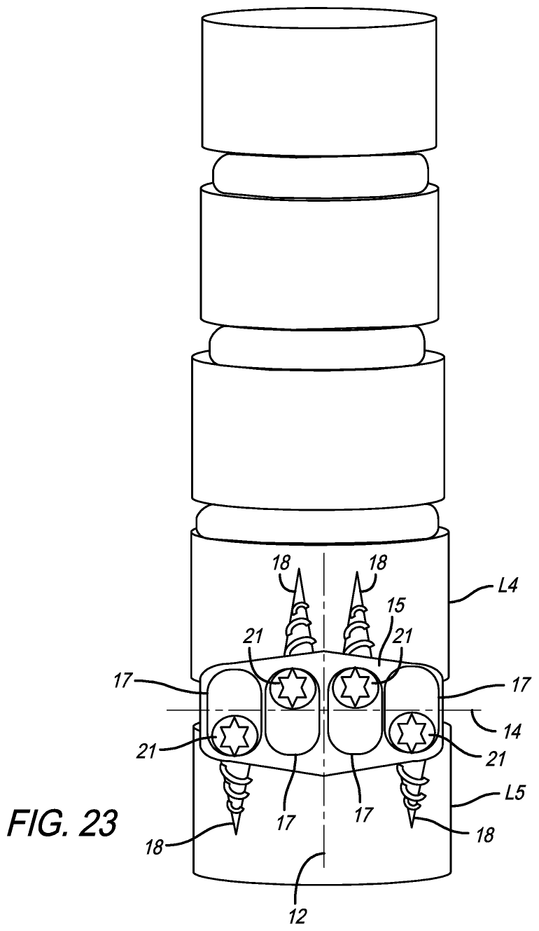

FIG. 22 shows a 3×2 cage body 11 attached to a patient's lumbar vertebrae L4 and L5 using three anchors, one anchor inserted into in each of the anchor apertures in column 1, row 2; column 2, row 1; and column 3, row 2. FIG. 23 shows a 4×2 cage body 11 attached to a patient's lumbar vertebrae L4 and L5 using three anchors, one anchor inserted into in each of the anchor apertures in column 1, row 2; column 2, row 1; and column 3, row 2.

The anchor apertures are configured to receive an anchor of a specific type and size. Anchor types include screws, self-locking screws, barbs, and curved anchoring barbs. In one embodiment the cage body 11 is secured with anchors having a 5.0 mm diameter anchor head, and in a preferred embodiment the cage body 11 is secured with friction-locking screws having a 5.5 mm diameter anchor head. Smaller anchors may be used, particularly in cage bodies having more than eight anchor apertures. For example, 5.0 mm diameter anchorhead anchors are known. A friction-lock anchor is shown in the figures throughout, but other types of anchors will suffice.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments falling within the scope of the appended claims.

1. An anterior lumbar interbody fusion (“ALIF”) device comprising:
a) a cage body having at least two anchor apertures therethrough, each anchor aperture configured to receive two anchors, each anchor having an anchor head; wherein
b) each anchor aperture has a proximal opening and a distal opening;
c) the proximal opening is a proximal obround comprising two semicircles of radiusₚ connected by two parallel lines each of the lengthₚ;
d) the distal opening is a distal obround comprising two semicircles of radius_{d} connected by two parallel lines each of the same length_{d}; and
e) radiusₚ is greater than radius_{d};
such that the anchor head having radius_{anchorhead} greater than radius_{d} can pass through the proximal opening but cannot pass through the distal opening; and
wherein the cage body is configured to be anchored to a first lumbar vertebra of a patient with fewer anchors than the number of anchor apertures.

2. The ALIF device of claim 1 wherein:
a) the cage body further comprises a cage vertical midline and a cage horizontal midline which intersect at a center point; and
b) each of the anchor apertures is disposed in the cage body at a different angle relative to the cage vertical midline.

3. The ALIF device of claim 1 wherein:
a) the cage body further comprises a cage vertical midline and a cage horizontal midline which intersect at a center point; and
b) at least one of the anchor apertures is disposed in the cage body at an angle relative to the cage horizontal midline that is different from that of at least one of the other anchor apertures.

4. The ALIF device of claim 1 wherein:
a) the proximal opening has lengthₚ where lengthₚ is at least (4×radius_{anchorhead});
b) the distal opening has length_{d} where length_{d} is at least (4×radius_{anchorhead}); and
c) lengthₚ is less than length_{d}.

5. The ALIF device of claim 1 wherein the device further comprises:
a) a first anchor and a second anchor; and
b) a first anchor aperture and a second aperture in the cage body; wherein
i. the cage body is configured to be anchored to a first lumbar vertebra of a patient with only the first anchor in the first anchor aperture; and
ii. the cage body is configured to be anchored to a second lumbar vertebra of a patient with only the second anchor in the second anchor aperture.

6. canceled

7. canceled

8. The ALIF device of claim 1 wherein lengthₚ and length_{d} are less than or equal to 1 mm.

9. canceled

10. The ALIF device of claim 1 wherein:
a) the cage body further comprises a cage vertical midline and a cage horizontal midline which intersect at a center point; and
b) each of the anchor apertures is disposed in the cage body at a different angle relative to the cage vertical horizontal midline.

11. canceled

12. canceled

13. An anterior lumbar interbody fusion (“ALIF”) device comprising:
a) a cage body having at least two anchor apertures therethrough, each anchor aperture configured to receive two anchors, each anchor having an anchor head; wherein the cage body further comprises a cage vertical midline and a cage horizontal midline which intersect at a center point; wherein
b) each anchor aperture has a proximal opening and a distal opening;
c) the proximal opening is a proximal obround comprising two semicircles of radiusₚ connected by two parallel lines each of the length₁;
d) the distal opening is a distal obround comprising two semicircles of radius_{d} connected by two parallel lines each of the length₂; and
e) radiusₚ is greater than radius_{d};
such that the anchor head having radius_{anchorhead} greater than radius_{d} cannot pass through the distal opening; and
wherein the cage body is configured to be anchored to a first lumbar vertebra of a patient with fewer anchors than the number of anchor apertures.

14. canceled

15. The ALIF device of claim 13 wherein:
a) each of the anchor apertures is disposed in the cage body at a different angle relative to the cage vertical midline.

16. canceled

17. The ALIF device of claim 13 wherein each of the anchor apertures is disposed in the cage body at a different angle relative to the cage horizontal midline.

18. canceled

19. canceled

20. canceled

21. canceled

22. The ALIF device of claim 13 further comprising a first anchor and a second anchor wherein the first anchor and second anchor are disposed in the same anchor aperture.

23. The ALIF device of claim 1 such that each anchor aperture permits each anchor to be inserted at a desired yaw and at a desired angle between 25-45 degrees out of the cage.

24. The ALIF device of claim 1 wherein both anchors are disposed in the same anchor aperture.

25. The ALIF device of claim 1 wherein:
a) the cage body further comprises a first anchor aperture and a second anchor aperture;
b) the cage body is configured to be anchored to a first lumbar vertebra of a patient with only one anchor in the first anchor aperture; and
c) the cage body is configured to be anchored to a second lumbar vertebra of a patient with only one anchor in the second anchor aperture.

26. The ALIF device of claim 1 further comprising:
a) a first anchor aperture, a second anchor aperture, and a third anchor aperture; and
b) the cage body is configured to be anchored to two adjacent lumbar vertebra of a patient with two anchors in the first anchor aperture, two anchors in the second anchor aperture, and only one anchor in the third anchor aperture.

27. The ALIF device of claim 1 further comprising:
a) a first anchor aperture, a second anchor aperture, and a third anchor aperture; and
b) the cage body is configured to be anchored to two adjacent lumbar vertebra of a patient with two anchors in the first anchor aperture, only one anchor in the second anchor aperture, and only one anchor in the third anchor aperture.

28. The ALIF device of claim 13 wherein the cage body is configured to be fixed to two lumbar vertebra of a patient with sufficient anchors to prevent skew about the center point or rotation about the vertical and horizontal midlines.

29. The ALIF device of claim 13 such that each anchor aperture permits each anchor to be inserted at a desired yaw and at a desired angle between 25-45 degrees out of the cage.

30. The ALIF device of claim 13 wherein both anchors are disposed in the same anchor aperture.

31. The ALIF device of claim 13 wherein:
a) the cage body further comprises a first anchor aperture and a second anchor aperture;
b) the cage body is configured to be anchored to a first lumbar vertebra of a patient with only one anchor in the first anchor aperture; and
c) the cage body is configured to be anchored to a second lumbar vertebra of a patient with only one anchor in the second anchor aperture.

32. The ALIF device of claim 13 further comprising:
a) a first anchor aperture, a second anchor aperture, and a third anchor aperture; and
b) the cage body is configured to be anchored to two adjacent lumbar vertebra of a patient with two anchors in the first anchor aperture, two anchors in the second anchor aperture, and only one anchor in the third anchor aperture.

33. The ALIF device of claim 13 further comprising:
a) a first anchor aperture, a second anchor aperture, and a third anchor aperture; and
b) the cage body is configured to be anchored to two adjacent lumbar vertebra of a patient with two anchors in the first anchor aperture, only one anchor in the second anchor aperture, and only one anchor in the third anchor aperture.
